(19) United States
(12) Patent Application Publication
(10) Pub. No.: US 2024/0418636 A1
(43) Pub. Date: Dec. 19, 2024

(54) Gas Absorption Spectrometer

(71) Applicant: SHIMADZU CORPORATION, Kyoto-shi (JP)

(72) Inventors: Yuta KAWASHIMA, Kyoto-shi (JP); Kazune MANO, Kyoto-shi (JP); Junki SAKAMOTO, Kyoto-shi (JP); Koji TOJO, Kyoto-shi (JP); Tetsuo FURUMIYA, Kyoto-shi (JP)

(21) Appl. No.: 18/676,822

(22) Filed: May 29, 2024

(30) Foreign Application Priority Data
Jun. 16, 2023 (JP) 2023-098928

(51) Int. Cl. G01N 21/31; G01N 33/00

(52) U.S. Cl. CPC G01N 21/31; G01N 33/0027; G01N 2201/06113; G01N 2201/0636

(57) ABSTRACT

A gas absorption spectrometer includes a resonator including a mirror, a laser light source that emits laser beams for irradiation of the resonator, a photodetector that detects light emitted from the resonator, a transmissive-type optical diffuser arranged in an optical path, the optical path being defined between the resonator and the photodetector, and a controller that measures a target component in gas present in the resonator based on an output signal from the photodetector. The optical diffuser has a first surface facing the resonator in the optical path and a second surface facing the photodetector in the optical path. The first surface is a flat surface and the second surface is a non-flat surface, the non-flat surface having bumps and dips for light scattering.

### CROSS REFERENCE TO RELATED APPLICATIONS

This nonprovisional application is based on Japanese Patent Application No. 2023-098928 filed with the Japan Patent Office on Jun. 16, 2023, the entire contents of which are hereby incorporated by reference.

### BACKGROUND OF THE INVENTION

### Field of the Invention

The present disclosure relates to a gas absorption spectrometer that determines a concentration of a target component in gas by cavity ring-down absorption spectroscopy (CRDS) which represents gas absorption spectroscopy.

### Description of the Background Art

Cavity ring-down absorption spectroscopy (CRDS) representing gas absorption spectroscopy has been known. CRDS refers to a spectroscopic technique for high-sensitivity determination of a concentration of a target component contained in gas, by extending an effective optical path length for light absorption by gas with the use of a resonator (cavity) including a highly reflective mirror. For example, documents below disclose a gas absorption spectrometer based on CRDS.
Technical Note “A survey on high-efficiency measurement techniques of trace moisture in gases,” Koji Hashiguchi, AIST Bulletin of Metrology, Vol. 9, No. 2, October 2015
“Development of a low-temperature cavity Ring-Down Spectrometer for the detection of Carbon-14,” McCartt, Stanford University, July 2014
“Adjacent-resonance etalon cancellation in ring-down spectroscopy,” Bradley M. Gibson, Optics Letters Vol. 43, No. 14, pp. 3257-3260 (2018)

In CRDS, light (laser beams) inputted to the resonator after light is stored in the resonator is cut off, and attenuation of light that leaks from the resonator after light is cut off is measured by the photodetector. By determining a time constant (ring-down time) of attenuation of light from measurement data, a concentration of a target component contained in gas in the resonator is measured.

### SUMMARY OF THE INVENTION

In gas absorption spectroscopic measurement with the use of the highly reflective mirror as in CRDS, what is called fringe noise may be caused by return light which is light reflected at a surface other than a reflection surface of the highly reflective mirror included in the resonator and returning into the resonator. Return light which becomes a factor for fringe noise may be produced at a plurality of locations, and it is one of factors that restrict sensitivity.

This invention was made to solve such a problem, and an object thereof is to lessen fringe noise in a gas absorption spectrometer including a highly reflective mirror based on cavity ring-down spectroscopy or the like.

A gas absorption spectrometer according to the present disclosure is a gas absorption spectrometer that measures a gas component, and includes a resonator including at least two mirrors, a light source that emits laser beams for irradiation of the resonator, a photodetector that detects light emitted from the resonator, at least one light transmissive member in a form of a plate arranged in an optical path, the optical path being defined between the resonator and the photodetector, and a control device that measures a target component in gas present in the resonator based on an output signal from the photodetector. The light transmissive member has a first surface facing the resonator in the optical path and a second surface facing the photodetector in the optical path. At least one of the first surface and the second surface is a non-flat surface, the non-flat surface having bumps and dips for light scattering.

The foregoing and other objects, features, aspects and advantages of this invention will become more apparent from the following detailed description of this invention when taken in conjunction with the accompanying drawings.

### BRIEF DESCRIPTION OF THE DRAWINGS

FIG. 1 is a diagram (No. 1) schematically showing a configuration of a gas absorption spectrometer.

FIG. 2 is a conceptual diagram for illustrating a mode frequency.

FIG. 3 is a diagram schematically showing an optical path in a configuration in a comparative example.

FIG. 4 is a diagram schematically showing an optical path in the gas absorption spectrometer.

FIG. 5 is a diagram (No. 1) showing an exemplary result of measurement of a baseline.

FIG. 6 is a diagram (No. 2) schematically showing a configuration of a gas absorption spectrometer.

FIG. 7 is a diagram (No. 2) showing an exemplary result of measurement of a baseline.

FIG. 8 is a diagram (No. 3) schematically showing a configuration of a gas absorption spectrometer.

FIG. 9 is a diagram (No. 4) schematically showing a configuration of a gas absorption spectrometer.

FIG. 10 is a diagram (No. 5) schematically showing a configuration of a gas absorption spectrometer.

### DESCRIPTION OF THE PREFERRED EMBODIMENTS

The present embodiment will be described below in detail with reference to the drawings. The same or corresponding elements in the drawings have the same reference characters allotted below and description thereof will not be repeated.

### <Configuration of Gas Absorption Spectrometer>

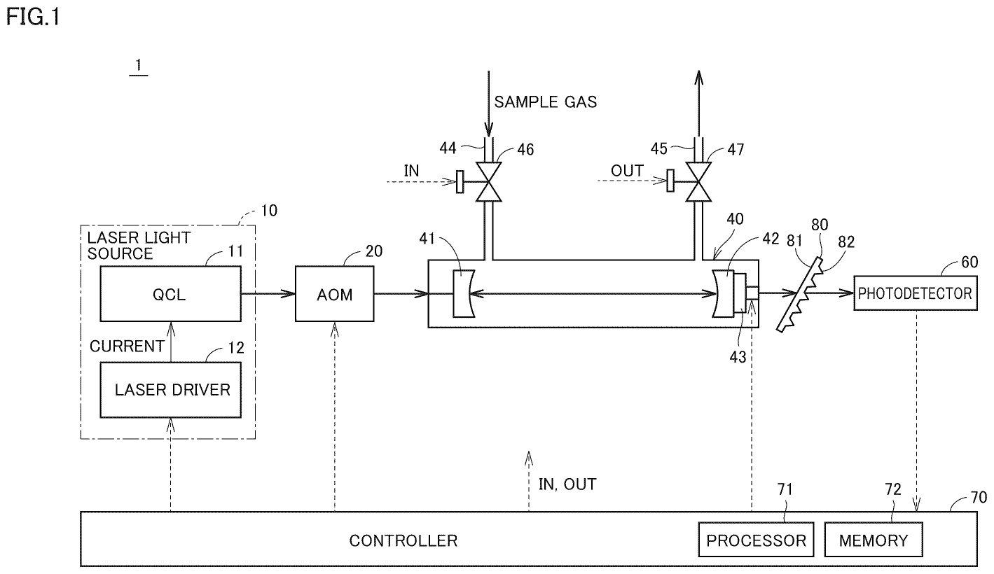

FIG. 1 is a diagram schematically showing a configuration of a gas absorption spectrometer 1 according to the present embodiment. Gas absorption spectrometer 1 is configured to measure by cavity ring-down absorption spectroscopy (CRDS), light absorption by a target component contained in sample gas.

Gas absorption spectrometer 1 includes a laser light source 10, an acousto-optic modulator (AOM) 20, a resonator 40, a photodetector 60, a controller 70, and a transmissive-type optical diffuser 80.

Laser light source 10 emits laser beams for irradiation of resonator 40. Laser light source 10 is configured as being variable in oscillation frequency of laser beams in accordance with an instruction from controller 70. Specifically, laser light source includes distributed feedback quantum cascade laser (QCL) 11 and a laser driver 12. QCL 11 emits laser beams, for example, around a central oscillation wave number of 2200 cm⁻¹ (around a wavelength of 4.5 μm). Laser driver 12 supplies a drive current to QCL 11 in accordance with an instruction from controller 70. By varying a drive current to QCL 11, an oscillation wave number of QCL 11 can be swept by approximately 0.2 cm⁻¹.

AOM 20 is provided in an optical path between laser light source 10 and resonator 40. AOM 20 is an optical switch (switch) that switches between emission and cut-off of laser beams from laser light source 10 to resonator 40 at a high speed in accordance with an instruction from controller 70. AOM 20 is set to an on state in which laser beams from laser light source 10 are outputted to resonator 40, by application of an on instruction for emission of light from controller 70. AOM 20 is set to an off state in which laser beams from laser light source 10 are not outputted to resonator 40, by application of an off instruction for cut-off from light from controller 70.

Resonator 40 is provided in an optical path between AOM 20 and photodetector 60. Resonator 40 includes a container (cell) in which sample gas can hermetically be sealed, and includes an introduction pipe 44 for introduction of sample gas into the inside before start of measurement and a discharge pipe 45 for discharge of sample gas to the outside after the end of measurement. Introduction pipe 44 is provided with an introduction valve 46. Discharge pipe 45 is provided with a discharge valve 47. Opening and closing of introduction valve 46 and discharge valve 47 can also be controlled by controller 70.

In the inside of resonator 40, a pair of mirrors 41 and 42 is provided. Mirrors 41 and 42 are arranged as being opposed to each other so as to reflect light therebetween in the inside of resonator 40. In order to easily satisfy a condition for stabilization of resonator 40, a concave mirror is adopted for each of mirrors 41 and 42. A mirror high in reflectivity (for example, around 99.9%) is adopted as each of mirrors 41 and 42 such that light that leaks to the outside of resonator 40 is extremely weak. A resonator length of resonator 40 (a distance between mirrors 41 and 42 in a direction of an optical axis) is, for example, approximately 450 mm. The number of mirrors to be arranged in resonator 40 is not limited to two, and three or more mirrors may be provided. In other words, a resonator in which mirrors are arranged such that light is reflected among them or a resonator in which mirrors are arranged in a ring such that light is reflected in one direction may be applicable.

A piezo element (piezoelectric element) 43 is arranged in mirror 42. Piezo element 43 displaces mirror 42 included in resonator 40 in the direction of the optical axis by driving mirror 42 in accordance with an instruction from controller 70. The resonator length of resonator 40 can thus be varied. Therefore, the resonator length is variable so as to coincide with a laser wave number, or the laser wave number can be swept so as to coincide with the resonator length. The piezo element may be arranged in mirror 41 rather than in mirror 42, or the piezo element may be arranged in both of mirror 41 and mirror 42.

Optical diffuser 80 is provided in the optical path between resonator 40 and photodetector 60. Optical diffuser 80 will be described in detail later.

Photodetector 60 is a photodetector such as a photodiode or an image sensor. Photodetector 60 detects as output light from resonator 40, weak light that has been taken out of mirror 42 in resonator 40 and passed through optical diffuser 80, and provides a signal (detection signal) indicating a result of detection to controller 70. For example, a liquid nitrogen cooled indium antimony (InSb) detector can be adopted as photodetector 60.

Controller 70 includes a processor 71 such as a central processing unit (CPU) or a field-programmable gate array (FPGA), a memory 72 such as a read only memory (ROM) and a random access memory (RAM), and an input and output port (not shown).

Controller 70 controls each device included in gas absorption spectrometer 1. Specifically, controller 70 provides an instruction for sweep of an oscillation frequency of laser beams to laser driver 12 or provides an on signal or an off signal described above to AOM 20. Controller 70 provides an instruction for introduction of sample gas into the inside of resonator 40 to introduction valve 46 or provides an instruction for discharge of sample gas to the outside of resonator 40 to discharge valve 47. Controller 70 has a voltage for displacement of mirror 42 applied to piezo element 43. Controller 70 performs various types of data processing for calculating a concentration (an absolute concentration) of a target component contained in sample gas based on a detection signal from photodetector 60.

Controller 70 may be configured as being divided into two or more units for each function. For example, controller 70 may be divided into a unit that controls each device and a unit that performs various types of data processing.

### <Principles of Measurement by Cavity Ring-Down Spectroscopy (CRDS)>

Principles in measurement by cavity ring-down absorption spectroscopy in gas absorption spectrometer 1 will briefly be described. In general, for a resonator, there is a resonance condition under which resonance occurs when a frequency of light emitted to the resonator has a specific frequency. A frequency of laser beams emitted to resonator 40 is called a “laser frequency” below. A frequency of light at which resonance in resonator 40 may occur is called a “mode frequency.”

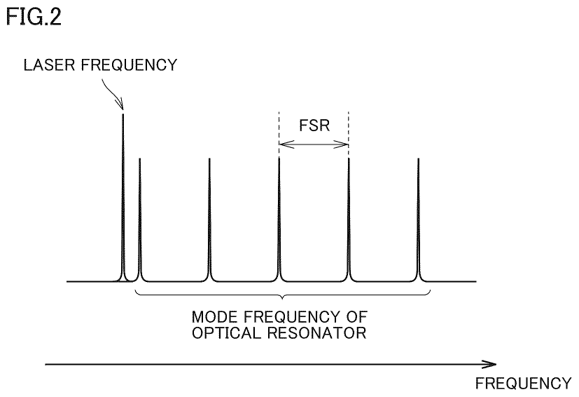

FIG. 2 is a conceptual diagram for illustrating a mode frequency. As shown in FIG. 2, mode frequencies are present in a prescribed frequency range. A range between two adjacent mode frequencies of a plurality of mode frequencies is referred to as a “free spectral range” (FSR) below.

When the laser frequency is not equal to any mode frequency, power of light is not stored in resonator 40. When the laser frequency is equal to any mode frequency, power of light is stored in resonator 40.

Controller 70 determines whether or not power of laser beams is sufficiently stored in resonator 40 based on the output signal from photodetector 60 (output light from resonator 40). Controller 70 determines that power of laser beams has sufficiently been stored in resonator 40 when output light from resonator 40 attains to a predetermined threshold value, and outputs the off signal to AOM 20. Light inputted to resonator 40 is thus cut off by AOM 20. Then, light stored in resonator 40 travels between mirror 41 and mirror 42 a large number of times (normally, several thousand to several ten thousand times). This light gradually attenuates due to loss caused by leakage by reflection by mirrors 41 and 42 and absorption by a target component in sample gas while light travels between mirrors 41 and 42. Therefore, output light from resonator 40 that leaks from mirror 42 gradually attenuates. According to CRDS, by increasing a distance (an effective optical path length) of passage of light through sample gas with the use of resonator 40, light absorption can be detected even though light absorption by the target component is very little.

Controller 70 obtains an output signal from photodetector 60 after AOM 20 cuts off light inputted to resonator 40 as a “ring-down signal” and calculates a time constant of attenuation of the obtained ring-down signal as “ring-down time.” Controller 70 calculates a concentration of the target component contained in sample gas from the calculated ring-down time.

Controller 70 obtains the output signal from photodetector 60, for example, at 0.2-μsec intervals, and calculates the ring-down time from the obtained output signal from photodetector 60. When there is no gas component that absorbs laser beams in the inside of resonator 40, the ring-down time is equal to the time constant of attenuation by resonator 40 and hence it has substantially a constant value. When there is a gas component that absorbs laser beams in the inside of resonator 40, on the other hand, the ring-down time has a value varied with a concentration of the gas component. The concentration of the target component can be quantified by using this feature.

### <Lessening of Fringe Noise>

In gas absorption spectroscopic measurement with the use of the highly reflective mirror as in CRDS, what is called fringe noise is caused by return light which is light reflected at a surface other than reflection surfaces of mirrors 41 and 42 in resonator 40 and returning into the resonator.

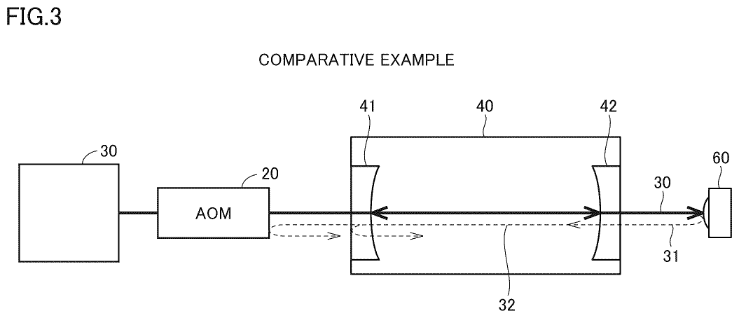

FIG. 3 is a diagram schematically showing an optical path in a configuration in a comparative example. FIG. 3 shows as the comparative example as compared to the present embodiment, an optical path in a configuration obtained by removal of optical diffuser 80 from gas absorption spectrometer 1.

As shown in FIG. 3, some of output light 30 from resonator 40 is reflected at a light receiving surface of photodetector 60 and returns to resonator 40 as reflected light 31. Return light 32 which is reflected light 31 returning into resonator 40 becomes stray light in resonator 40. Consequently, noise (fringe noise) is superimposed on a baseline of an absorption spectrum (a result of output from photodetector 60 when vacuum is produced in resonator 40) and accuracy in spectroscopy may lower.

In view of points above, in gas absorption spectrometer 1 according to the present embodiment, transmissive-type optical diffuser 80 in a form of a plate is provided in the optical path between resonator 40 and photodetector 60. Optical diffuser 80 is configured to diffuse light 31 reflected at the light receiving surface of photodetector 60 while it allows passage of output light 30 from resonator 40 therethrough.

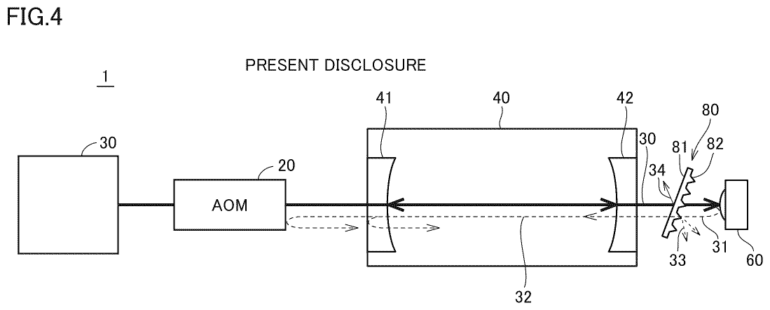

FIG. 4 is a diagram schematically showing an optical path in gas absorption spectrometer 1 according to the present embodiment. As described above, optical diffuser 80 is provided in the optical path between resonator 40 and photodetector 60.

Optical diffuser 80 has a first surface facing resonator 40 and a second surface facing photodetector 60 in the optical path of output light 30 from resonator 40. The first surface of optical diffuser 80 (the surface on a side of resonator 40) is a flat surface (even surface) 81 through which light passes without being scattered. The second surface of optical diffuser 80 (the surface on a side of photodetector 60) is a non-flat surface 82 provided with fine projections and recesses (surface roughness) which scatter light. Surface roughness (size of projections and recesses) of non-flat surface 82 is not particularly limited so long as roughness is sufficient for scattering of light.

Optical diffuser 80 can be made of opaque glass (frosted glass) provided with fine projections and recesses in one surface, the projections and recesses being provided, for example, by subjecting one surface of transparent plate glass to working called sand blasting.

In gas absorption spectrometer 1 according to the present embodiment, some of light 31 reflected at the light receiving surface of photodetector 60 is irregularly reflected and diffused at non-flat surface 82 of optical diffuser 80. Consequently, an amount of return light 32 that enters the inside of resonator 40 decreases and fringe noise is lessened.

Some of output light 30 is reflected at flat surface 81 of optical diffuser 80 without passing through optical diffuser 80. In gas absorption spectrometer 1 according to the present embodiment, flat surface 81 of optical diffuser 80 is arranged as being inclined with respect to a plane orthogonal to the optical path of output light 30. Therefore, return of light 34 reflected at flat surface 81 to resonator 40 can more readily be suppressed. Fringe noise can thus more readily be lessened.

Though fringe noise derived from return light 32 can also be lessened by arrangement of an optical isolator in the optical path between resonator 40 and photodetector 60, the isolator is greater in loss in a forward direction and more expensive than optical diffuser 80 (frosted glass or the like). Therefore, by providing transmissive-type optical diffuser 80 in the optical path between resonator 40 and photodetector 60 as in the present embodiment, fringe noise can be lessened with less loss and lower cost.

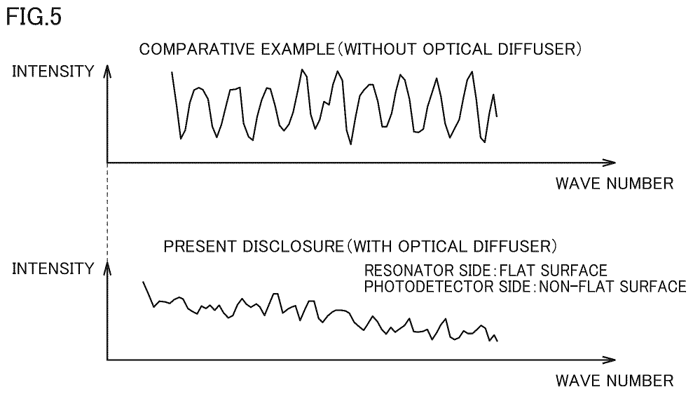

FIG. 5 is a diagram showing an exemplary result of measurement of a baseline in an example where a gas absorption spectrometer without an optical diffuser (comparative example) is used and an exemplary result of measurement of a baseline in an example where gas absorption spectrometer 1 with optical diffuser 80 (present embodiment) is used. In FIG. 5, the abscissa represents a wave number of laser beams and the ordinate represents measured intensity of an output signal from photodetector 60.

In the example without the optical diffuser (comparative example), relatively great fringe noise is periodically superimposed on the wave number. This fringe noise may greatly be affected by return light 32 which results from some of light that goes out of resonator 40 being reflected at the light receiving surface of photodetector 60 and returning into resonator 40.

In contrast, in the example with optical diffuser 80 (present embodiment), it can be understood that magnitude of fringe noise has been made smaller. This may be because the amount of return light 32 that enters the inside of resonator 40 decreases as a result of diffusion by optical diffuser 80, of some of light reflected at the light receiving surface of photodetector 60 as described above.

As set forth above, in gas absorption spectrometer 1 according to the present embodiment, light transmissive-type optical diffuser 80 is arranged in the optical path between resonator 40 and photodetector 60. The second surface (the surface on the side of photodetector 60) of optical diffuser 80 is non-flat surface 82 provided with fine projections and recesses which scatter light. Some of light that goes out of resonator and is reflected at the light receiving surface of photodetector 60 can thus be diffused by optical diffuser 80 and the amount of return light 32 that returns into resonator 40 can be decreased. Consequently, fringe noise can be lessened.

### First Modification

Though the first surface (the surface on the side of resonator 40) of optical diffuser 80 is flat surface 81 and the second surface (the surface on the side of photodetector 60) is non-flat surface 82 in the embodiment described above, the first surface of optical diffuser 80 may be non-flat surface 82 and the second surface may be flat surface 81.

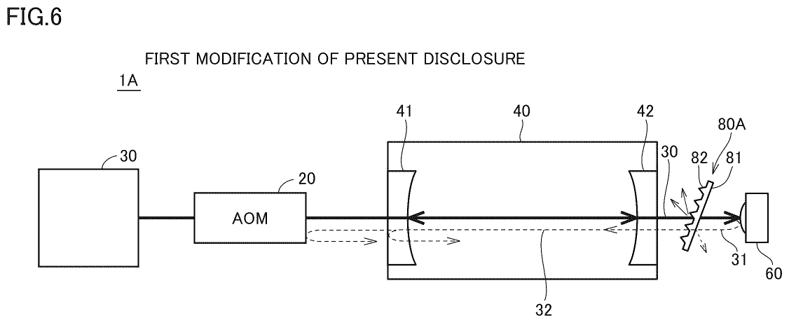

FIG. 6 is a diagram schematically showing a configuration of a gas absorption spectrometer 1A according to the present first modification. Gas absorption spectrometer 1A includes an optical diffuser 80A that replaces optical diffuser 80 in gas absorption spectrometer 1 described above. The first surface (the surface on the side of resonator 40) of optical diffuser 80A is non-flat surface 82 and the second surface (the surface on the side of photodetector 60) of optical diffuser 80A is flat surface 81.

In gas absorption spectrometer 1A, some of output light 30 from resonator 40 is irregularly reflected and scattered at non-flat surface 82 of optical diffuser 80 without passing through optical diffuser 80. Some of light 31 reflected at the light receiving surface of photodetector 60 is reflected at flat surface 81 without passing through optical diffuser 80. Since the amount of return light 32 that enters the inside of resonator 40 can thus be decreased, fringe noise can be lessened.

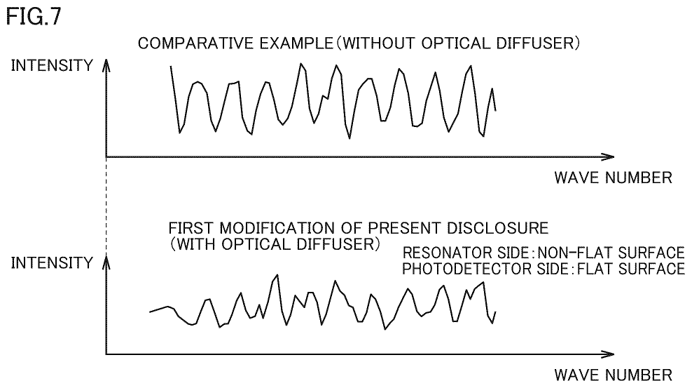

FIG. 7 is a diagram showing an exemplary result of measurement of a baseline in the example where the gas absorption spectrometer without the optical diffuser (comparative example) is used and an exemplary result of measurement of a baseline in an example where gas absorption spectrometer 1A with optical diffuser 80A (present first modification) is used.

In the example without the optical diffuser (comparative example), relatively great fringe noise is periodically superimposed on the wave number.

In contrast, in the example with optical diffuser 80A (present first modification), it can be understood that magnitude of fringe noise has been made smaller. This may be because the amount of return light 32 can be decreased as a result of diffusion and reflection of light by optical diffuser 80 as described above.

As set forth above, optical diffuser 80A provided with the first surface (the surface on the side of resonator 40) which is flat surface 82 and the second surface (the surface on the side of photodetector 60) which is flat surface 81 may be arranged in the optical path between resonator 40 and photodetector 60.

When fringe noise in the present first modification is compared with fringe noise in the embodiment described above, the former is slightly greater than the latter.

This may be because some of light scattered at non-flat surface 82 on the side of resonator 40 of optical diffuser 80A returns into resonator 40 in addition to return light 32 reflected at the light receiving surface of photodetector 60.

### Second Modification

Though the first surface (the surface on the side of resonator 40) of optical diffuser 80A according to the first modification described above is non-flat surface 82, in this case, the first surface of optical diffuser 80A does not necessarily have to be inclined with respect to the plane orthogonal to the optical path of output light 30.

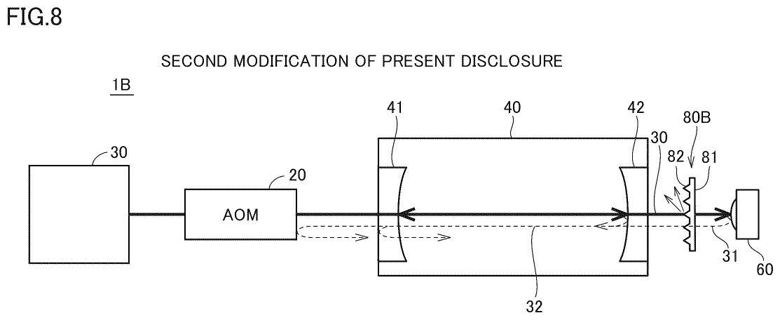

FIG. 8 is a diagram schematically showing a configuration of a gas absorption spectrometer 1B according to the present second modification. Gas absorption spectrometer 1B includes an optical diffuser 80B that replaces optical diffuser 80A of gas absorption spectrometer 1A described above.

The first surface (the surface on the side of resonator 40) of optical diffuser 80B is non-flat surface 82 and the second surface (the surface on the side of photodetector 60) of optical diffuser 80B is flat surface 81. Furthermore, the first surface (non-flat surface 82) of optical diffuser 80B is orthogonal to the optical path of output light 30. In gas absorption spectrometer 1B, some of output light 30 is irregularly reflected and scattered at the first surface (non-flat surface 82) of optical diffuser 80B without passing through optical diffuser 80B. Though some of light scattered at the first surface (non-flat surface 82) of optical diffuser 80B returns into resonator 40, an amount of light that returns into resonator 40 may substantially be constant regardless of an angle of inclination of the first surface of optical diffuser 80B. Therefore, even when the first surface (non-flat surface 82) of optical diffuser 80B is orthogonal to the optical path of output light 30 as in the present second modification, fringe noise can be lessened as in the first modification described above.

### Third Modification

Of the first surface (the surface on the side of resonator 40) and the second surface (the surface on the side of photodetector 60) of optical diffuser 80, only the second surface is non-flat surface 82 in the embodiment described above. Both of the first surface and the second surface of the optical diffuser, however, may be non-flat surfaces 82.

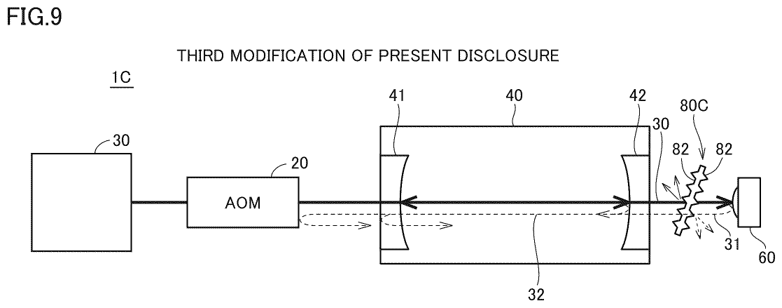

FIG. 9 is a diagram schematically showing a configuration of a gas absorption spectrometer 1C according to the present third modification. Gas absorption spectrometer 1C includes an optical diffuser 80C that replaces optical diffuser 80 in gas absorption spectrometer 1 described above. Optical diffuser 80C has the first surface and the second surface both provided as non-flat surfaces 82.

In gas absorption spectrometer 1C, some of output light 30 is irregularly reflected and scattered at the first surface (non-flat surface 82) of optical diffuser 80C without passing through optical diffuser 80C and some of light 31 reflected at the light receiving surface of photodetector 60 is also irregularly reflected and scattered at the second surface (non-flat surface 82) of optical diffuser 80C. Consequently, the amount of return light 32 that returns into resonator 40 can be decreased and fringe noise can be lessened.

### Fourth Modification

Though a single optical diffuser 80 is provided in the embodiment described above, two or more optical diffusers 80 may be provided.

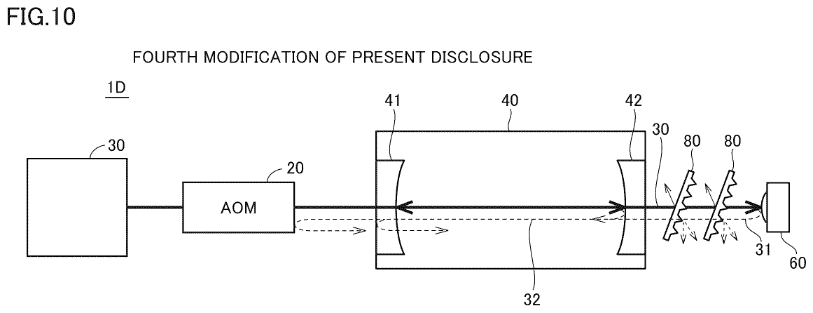

FIG. 10 is a diagram schematically showing a configuration of a gas absorption spectrometer 1D according to the present fourth modification. In gas absorption spectrometer 1D, two optical diffusers 80 described above are arranged in the optical path between resonator 40 and photodetector 60. In gas absorption spectrometer 1D, more return light 32 can be lessened owing to arrangement of two optical diffusers 80. Consequently, fringe noise can further be lessened.

### Aspects

The embodiment and the modifications thereof described above will be understood by a person skilled in the art as specific examples of aspects below.

(Clause 1) A gas absorption spectrometer according to the present disclosure is a gas absorption spectrometer that measures a gas component, and includes a resonator including at least two mirrors, a light source that emits laser beams for irradiation of the resonator, a photodetector that detects light emitted from the resonator, at least one light transmissive member in a form of a plate arranged in an optical path, the optical path being defined between the resonator and the photodetector, and a control device that measures a target component in gas present in the resonator based on an output signal from the photodetector. The light transmissive member has a first surface facing the resonator in the optical path and a second surface facing the photodetector in the optical path. At least one of the first surface and the second surface is a non-flat surface, the non-flat surface having bumps and dips for light scattering.

In the gas absorption spectrometer described in Clause 1, at least one light transmissive member is arranged in the optical path between the resonator and the photodetector. At least one of the first surface (the surface facing the resonator) and the second surface (the surface facing the photodetector) of the light transmissive member is the non-flat surface having bumps and dips for light scattering. Some of light that goes out of the resonator and is reflected at the light receiving surface of the photodetector can be diffused at the non-flat surface of the light transmissive member to decrease an amount of return light that returns into the resonator. Consequently, fringe noise can be

(Clause 2) In the gas absorption spectrometer described in Clause 1, the second surface is the non-flat surface.

In the gas absorption spectrometer described in Clause 2, some of light that goes out of the resonator and is reflected at the light receiving surface of the photodetector can be diffused at the second surface (the surface facing the photodetector) of the light transmissive member to thereby decrease an amount of return light that returns into the resonator.

(Clause 3) In the gas absorption spectrometer described in Clause 2, the first surface is a flat surface inclined with respect to the optical path.

In the gas absorption spectrometer described in Clause 3, return of light that goes out of the resonator and is reflected at the first surface (the surface facing the resonator) of the light transmissive member to the resonator can more readily be suppressed. Fringe noise can thus more readily be lessened.

(Clause 4) In the gas absorption spectrometer described in Clause 2, the first surface is the non-flat surface.

In the gas absorption spectrometer described in Clause 4, an amount of return light that returns into the resonator can be decreased by scattering light at both of the first surface and the second surface of the light transmissive member.

(Clause 5) In the gas absorption spectrometer described in any one of Clauses 1 to 4, the control device measures the target component in gas by on cavity ring-down spectroscopy.

In the gas absorption spectrometer described in Clause 6, fringe noise in measurement of the target component in gas by cavity ring-down absorption spectroscopy can be lessened.

(Clause 6) The gas absorption spectrometer described in Clause 5 further includes a switch arranged between the light source and the resonator, the switch being switched between an on state in which laser beams from the light source are outputted to the resonator and an off state in which laser beams from the light source are not outputted to the resonator. The control device measures the target component based on the output signal from the photodetector after the switch is switched from the on state to the off state.

In the gas absorption spectrometer described in Clause 6, fringe noise superimposed on the output signal from the photodetector after the switch is switched from the on state to the off state can be lessened. Consequently, the target component in gas can accurately be measured.

Though an embodiment of the present invention has been described, it should be understood that the embodiment disclosed herein is illustrative and non-restrictive in every respect. The scope of the present invention is defined by the terms of the claims and is intended to include any modifications within the scope and meaning equivalent to the terms of the claims.

What is claimed is:

1. A gas absorption spectrometer that measures a gas component, the gas absorption spectrometer comprising:
a resonator including at least two mirrors;
a light source that emits laser beams for irradiation of the resonator;
a photodetector that detects light emitted from the resonator;
at least one light transmissive member in a form of a plate arranged in an optical path, the optical path being defined between the resonator and the photodetector; and
a control device that measures a target component in gas present in the resonator based on an output signal from the photodetector, wherein
the light transmissive member has
a first surface facing the resonator in the optical path, and
a second surface facing the photodetector in the optical path, and
at least one of the first surface and the second surface is a non-flat surface, the non-flat surface having bumps and dips for light scattering.

2. The gas absorption spectrometer according to claim 1, wherein
the second surface is the non-flat surface.

3. The gas absorption spectrometer according to claim 2, wherein
the first surface is a flat surface inclined with respect to the optical path.

4. The gas absorption spectrometer according to claim 2, wherein
the first surface is the non-flat surface.

5. The gas absorption spectrometer according to claim 1, wherein
the control device measures the target component in gas by cavity ring-down spectroscopy.

6. The gas absorption spectrometer according to claim 5, further comprising a switch arranged between the light source and the resonator, the switch being switched between an on state in which laser beams from the light source are outputted to the resonator and an off state in which laser beams from the light source are not outputted to the resonator, wherein
the control device measures the target component based on the output signal from the photodetector after the switch is switched from the on state to the off state.
